(19) United States
(12) Patent Application Publication
(10) Pub. No.: US 2024/0010608 A1
(43) Pub. Date: Jan. 11, 2024

(54) KETTLE-TYPE CONTINUOUS PRODUCTION METHOD FOR GLYCINE

(71) Applicant: HUAYANG NEW MATERIAL TECHNOLOGY GROUP CO., LT, Shanxi Province (CN)

(72) Inventors: Wei ZHANG, Shanxi Province (CN); Nan WU, Shanxi Province (CN); Ruiyuan HAO, Shanxi Province (CN); Xin LI, Shanxi Province (CN); Meng HAN, Shanxi Province (CN); Wenzhao GONG, Shanxi Province (CN); Renjun YANG, Shanxi Province (CN); Huimin HU, Shanxi Province (CN); Ying MENG, Shanxi Province (CN); Xue MEI, Shanxi Province (CN); Qiuhua YUAN, Shanxi Province (CN); Weibin LI, Shanxi Province (CN); Xuqing GUO, Shanxi Province (CN); Yanhui HAN, Shanxi Province (CN); Shaoke WANG, Shanxi Province (CN)

(21) Appl. No.: 17/610,458

(22) PCT Filed: Aug. 14, 2021
(86) PCT No.: PCT/CN2021/112637

§ 371 Date: Sep. 12, 2023

(30) Foreign Application Priority Data
Dec. 17, 2020 (CN) 202011500112.5

(51) Int. Cl. C07C 227/24; C07C 227/40

(52) U.S. Cl. CPC C07C 227/24; C07C 227/40

(57) ABSTRACT

Disclosed is a kettle-type continuous production method for glycine. The method includes: performing a hydantoin synthesis and hydrolysis reaction on glycolonitrile, ammonium carbonate, ammonium bicarbonate and water in a reactor with multiple serially connected kettles, and then purifying, concentrating, crystallizing, separating and drying the products to obtain refined glycine. The reactor with multiple serially connected kettles consists of a hydantoin synthesis section and a hydantoin hydrolysis section which are connected in sequence, the hydantoin synthesis section including a first reaction kettle group of reaction kettles with a reaction temperature of 80-100° C. and a second reaction kettle group of reaction kettles with a reaction temperature of 100-120° C., the first reaction kettle group or the second reaction kettle group consisting of one or more reaction kettles connected in series, the hydantoin hydrolysis section including a third reaction kettle group of reaction kettles with a reaction temperature of 130-150° C. and a fourth reaction kettle group of reaction kettles with a reaction temperature of 160-180° C., and the third reaction kettle group or the fourth reaction kettle group consisting of one or more reaction kettles connected in series.

The present invention claims the priority of the Chinese Patent Application No. 202011500112.5, filed to the Chinese Patent Office on Dec. 17, 2020, and entitled “KETTLE-TYPE CONTINUOUS PRODUCTION METHOD FOR GLYCINE”, which is incorporated herein by reference in its entirety.

### FIELD OF TECHNOLOGY

The present invention relates to the field of fine chemical industries, and in particular to a kettle-type continuous production method for glycine.

### BACKGROUND OF THE INVENTION

As the most basic amino acid, glycine is widely used in fields of pesticides, medicines, food, feed, daily chemicals, organic synthesis, etc. Since a market scale of glycine exceeds 350000 tons, China is the largest producer and consumer of glycine in the world. Current industrial production technologies of glycine mainly include an improved Strecker method, a direct hydantoin method or a chloroacetic acid ammonolysis method. Wherein, 1) A hydrolysis method includes: taking a natural protein such as gelatin or silk as a raw material, and performing hydrolyzing, separating, refining, filtering and drying to obtain glycine. 2) A chloroacetic acid method includes: dissolving a catalyst, urotropine, in ammonia water, dropwise adding a chloroacetic acid with thorough stirring at 30-50° C., after feeding, raising a temperature to 72-78° C., maintaining the temperature for 3 hours, then cooling, and finally recrystallizing twice with ethanol or methanol, to obtain glycine with purity of 95% or so. 3) A Strecker method includes: mixing a formaldehyde aqueous solution with sodium cyanide (or potassium cyanide) and ammonium chloride, enabling same to react at a low temperature, adding an acetic acid after a reaction to precipitate methylaminoacetonitrile, dissolving the methylaminoacetonitrile in ethanol, then adding a sulfuric acid to convert the methylaminoacetonitrile into aminoacetonitrile sulfate, then adding stoichiometric barium hydroxide, to generate barium sulfate and glycine, filtering same, and finally concentrating and crystallizing a filtrate to obtain glycine. 4) The improved Strecker method: in order to improve the quality of glycine and reduce a production cost and environmental pollution, the improved Strecker method using a hydrocyanic acid instead of sodium cyanide or potassium cyanide has been developed abroad. In a reaction, a hydrocyanic acid, formaldehyde, ammonia and carbon dioxide are used as raw materials, and a reaction liquid reacts in a tubular reactor. Glycine is precipitated at a low temperature, and a mother liquid is recycled. The concentration of a by-product in a reaction system is changed to move balance towards a target product, and therefore, a reaction yield is increased. 5) The direct hydantoin method: glycolonitrile is an addition product of a hydrocyanic acid and formaldehyde, stability of which is obviously improved compared with the hydrocyanic acid and its aqueous solution. The direct hydantoin method includes: taking glycolonitrile as a main raw material, enabling same to react with an ammonia source and a carbon source (ammonia water and carbon dioxide or ammonium bicarbonate) to synthesize hydantoin with thorough stirring at a certain temperature, then hydrolyzing the hydantoin at a certain temperature and pressure to obtain a glycine reaction liquid, and finally gas extracting, concentrating, decolorizing, crystallizing, separating, drying, etc. the glycine reaction liquid to obtain glycine, where a mother liquid is recycled. 6) An aminoacetonitrile method includes: enabling glycolonitrile to react with ammonia water to obtain aminoacetonitrile, adding an inorganic base for alkaline hydrolysis, adding an inorganic acid for neutralization to obtain a glycine reaction liquid, and finally concentrating, decoloring, crystallizing, separating, etc. the glycine reaction liquid to obtain glycine, where a mother liquid is recycled.

Owing to available raw material and low technical threshold, the chloroacetic acid method which was generally eliminated abroad is still used for producing glycine in China. Compared with the improved Strecker method and the direct hydantoin method, the method has a higher production cost, poorer product quality, heavier environmental pollution, etc. which are insurmountable.

Disclosed in the patent with the publication number of CN 107325015A is a method for continuously preparing glycine from glycolonitrile. The method utilizes the direct hydantoin method to prepare glycine, thereby continuously producing the glycine. Whereas, the method still has disadvantages. For example, the method uses a tubular packed reactor and a kettle type reactor connected in series as a core reaction device, but the reaction device has a high manufacturing cost, and undesirable efficiency. A method for processing a product obtained from a hydantoin hydrolysis reaction has high energy consumption and low efficiency. A pressure reaction vessel has low safety. In order to solve the problems, it is necessary to find a novel continuous production method for glycine as an alternative.

### BRIEF SUMMARY OF THE INVENTION

One aspect of the present invention provides a kettle-type continuous production method for glycine aiming at the problems of a high device cost, low efficiency, low safety, etc., in a method for continuously preparing glycine from glycolonitrile in the prior art.

A technical solution provided by the present invention is as follows:
a kettle-type continuous production method for glycine includes: performing a hydantoin synthesis and hydrolysis reaction on glycolonitrile, ammonium carbonate, ammonium bicarbonate and water in a reactor with multiple serially connected kettles, and then purifying, concentrating, crystallizing, separating and drying the products to obtain refined glycine,
where the reactor with multiple serially connected kettles consists of a hydantoin synthesis section and a hydantoin hydrolysis section which are connected in sequence,
the hydantoin synthesis section including a first reaction kettle group of reaction kettles with a reaction temperature of 80-100° C. and a second reaction kettle group of reaction kettles with a reaction temperature of 100-120° C., the first reaction kettle group or the second reaction kettle group consisting of one or more reaction kettles connected in series,
the hydantoin hydrolysis section including a third reaction kettle group of reaction kettles with a reaction temperature of 130-150° C. and a fourth reaction kettle group of reaction kettles with a reaction temperature of 160-180° C., and the third reaction kettle group or the fourth reaction kettle group consisting of one or more reaction kettles connected in series.

In the technical solution of the present invention, a pure kettle type series reactor (reactor with multiple serially connected kettles) is used, thereby further reducing an apparatus investment and improving reaction efficiency under the condition that advantages of a continuous production process for glycine are maintained.

In the present invention, a reaction equation formula of the kettle-type continuous production method for glycine is:

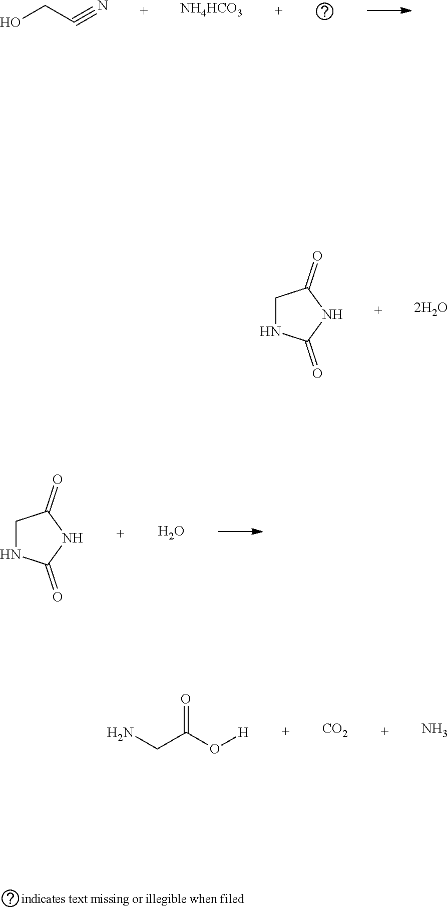

In the present invention, each reaction kettle of the reactor with multiple serially connected kettles may be connected to another reaction kettle in series in any suitable connection mode, for example, through a pipeline pump, an overflow pipe, etc. Preferably, in one embodiment of the present invention, each reaction kettle mentioned above is connected to another reaction kettle through the overflow pipe. Functional devices such as a pressure test device, a temperature test device and a flow rate test device may also be added on each kettle or between kettles mentioned above as required, which is deemed to fall within the scope of protection of the present invention.

In the present invention, the reaction kettle may be any suitable commercially available chemical reaction kettle including, but not limited to, a carbon steel reaction kettle, a stainless steel reaction kettle, a steel lined polyethylene (PE) reaction kettle, a steel lined polytetrafluoroethylene (PTEF) reaction kettle, a steel lined titanium reaction kettle, etc. Preferably, in one embodiment of the present invention, the reaction kettle in the hydantoin hydrolysis section uses urea-grade stainless steel as a kettle body lining. The urea-grade stainless steel kettle body lining may be arranged in the reaction kettle through any suitable method, or it is feasible to purchase a finished reaction kettle.

In the present invention, according to different reaction requirements, a plurality of reaction kettle groups may be added in the hydantoin synthesis section and the hydantoin hydrolysis section separately, to meet production requirements at different reaction temperatures, pressures and feeding amounts, which is deemed to fall within the scope of protection of the present invention. However, the hydantoin synthesis section shall at least include the first reaction kettle group of reaction kettles with the reaction temperature of 80-100° C. and the second reaction kettle group of reaction kettles with the reaction temperature of 100-120° C.; and the hydantoin hydrolysis section shall at least include the third reaction kettle group of reaction kettles with the reaction temperature of 130-150° C. and the fourth reaction kettle group of reaction kettles with the reaction temperature of 160-180° C., so as to ensure temperature or energy requirements of different reaction stages.

In the present invention, each reaction kettle group may include one or more sub-reaction kettles, and each sub-reaction kettle may be connected to another sub-reaction kettle in series through any suitable device. Preferably, in one embodiment of the present invention, the sub-reaction kettles are connected through an overflow pipe.

In the present invention, the reaction kettle of the reactor with multiple serially connected kettles may be set to any size according to a volume of the raw material, an effective volume of which may be the same or not. Preferably, in one embodiment of the present invention, the effective volumes of the reaction kettles of the reactor with multiple serially connected kettles are different, and the reaction kettle of the reactor with multiple serially connected kettles are sequentially connected from small effective volumes to large ones. It is possible to rationally assign retention times of a material liquid to different temperature sections when the reaction kettles are sequentially connected from small effective volumes to large ones.

Preferably, in one embodiment of the present invention, the inventor further uses a mode of sectioned feeding of glycolonitrile, ammonium carbonate, ammonium bicarbonate and water, which may further improve a solid content of a reaction liquid and reduce a subsequent water removal cost. That is, the ammonium carbonate, the ammonium bicarbonate and the water are proportionally mixed into a slurry, and the slurry enters the reactor with multiple serially connected kettles, and is subjected to a synthesis reaction with glycolonitrile which is introduced to the hydantoin synthesis section.

Preferably, in one embodiment of the present invention, the ammonium carbonate, ammonium bicarbonate and water are proportionally mixed into the slurry in a slurry mixing machine, and then the slurry is conveyed to the reactor with multiple serially connected kettles. More preferably, the ammonium carbonate, the ammonium bicarbonate and the water are preheated when mixed into the slurry.

Preferably, in one embodiment of the present invention, a mass ratio of the glycolonitrile between the first reaction kettle group and the second reaction kettle group meets that a ratio of the total feeding amount of the first reaction kettle group to the total feeding amount of the second reaction kettle group is 3-5:1. When the first reaction kettle group or the second reaction kettle group is composed of a plurality of reaction kettles, the feeding amount of the first reaction kettle group or the second reaction kettle group is the sum of feeding amounts of various reaction kettles.

Preferably, in one embodiment of the present invention, an amount-of-substance ratio of the glycolonitrile to the ammonium carbonate to the ammonium bicarbonate to the water is 1:1-2:2-3:20-30.

When the reaction kettles of the reactor with multiple serially connected kettles are sequentially connected from small effective volumes to large ones, preferably, in one embodiment of the present invention, a pressure and a retention time of the first reaction kettle group of reaction kettles are 3-7 MPa and 0.5-0.8 h respectively; a pressure and a retention time of the second reaction kettle group of reaction kettles are 3-7 MPa and 1.0-1.3 h respectively; a pressure and a retention time of the third reaction kettle group of reaction kettles are 3-7 MPa and 1.5-2.0 h respectively; and a pressure and a retention time of the fourth reaction kettle group of reaction kettles are 3-7 MPa and 2.5-3.0 h respectively.

Preferably, in one embodiment of the present invention, the purifying includes removing, in a deamination tower, ammonia from a product in the hydantoin hydrolysis section. Furthermore, the purifying may also include removing, in a flash tank, carbon dioxide from the product in the hydantoin hydrolysis section.

In the present invention, the purifying may be supplemented with any suitable step as required, to further remove an impurity in the product, which is deemed to fall within in the scope of protection of the present invention.

Preferably, in one embodiment of the present invention, carbon dioxide and ammonia generated through the purifying, evaporation condensate generated through the concentrating and a crystallization mother liquid generated through the crystallizing are used as raw materials for recycling.

More particularly, in one embodiment of the present invention, the method of the present invention may include:
mixing ammonium bicarbonate, ammonium carbonate and water proportionally in a slurry mixing machine, conveying, through a slurry pump, a slurry into a reactor with multiple serially connected kettles, and mixing same with introduced raw glycolonitrile for a reaction, where an amount-of-substance ratio of the glycolonitrile to the ammonium carbonate to the ammonium bicarbonate to the water is 1:1-2:2-3:20-30; and the reactor with multiple serially connected kettles is formed by sequentially connecting four reaction kettles from small effective volumes to large ones, a first kettle and a second kettle forming a hydantoin synthesis section, and a third kettle and a fourth kettle forming a hydantoin hydrolysis section, reaction temperatures, pressures and average retention times of material liquids of which are sequentially as follows: a temperature, a pressure and a retention time of the first reaction kettle are 80-100° C., 3-7 MPa and 0.5-0.8 h respectively, a temperature, a pressure and a retention time of the second reaction kettle are 100-120° C., 3-7 MPa and 1.0-1.3 h respectively, a temperature, a pressure and a retention time of the third reaction kettle are 130-150° C., 3-7 MPa and 1.5-2.0 h respectively, and a temperature, a pressure and a retention time of the fourth reaction kettle are 160-180° C., 3-7 MPa and 2.5-3.0 h respectively; and enabling a material liquid from the fourth reaction kettle to enter a flash tank to remove carbon dioxide through multi-stage flashing, then enabling a flashed material liquid to enter a deamination tower to remove ammonia and part of water, then enabling a material liquid subjected to ammonia and water removal to enter a triple-effect evaporator for evaporation and concentration until a large number of crystals appear (slurry), and finally primarily crystallizing, centrifugating, heating, dissolving, decoloring, filtering, secondarily crystallizing, centrifugating and drying the crystal to obtain a refined glycine product; and where the carbon dioxide and the ammonia removed through the flash tank and the deamination tower respectively are sent to a slurry machine by a batching kettle for batching and recycling; condensate generated through triple-effect evaporation is sent to the slurry machine for recycling; a crystallization mother liquid I is sent to the hydantoin hydrolysis section for recycling; a crystallization mother liquor II is sent to a dissolution kettle for recycling; and waste activate carbon is sent to a waste heat boiler for incineration.

According to the method, a continuous production method for preparing glycine from glycolonitrile is implemented. A flowchart of the method is as shown in FIG. 1.

The present invention has the beneficial effects:

Under the condition that the advantages of the continuous production process for glycine are maintained, the method of the present invention uses the pure kettle type series reactor, thereby further reducing the apparatus investment and reducing the subsequent water removal cost by introducing a mode of feeding an ammonium carbonate slurry and sectioned feeding of the glycolonitrile. The reaction kettle of the hydrolysis section uses the urea-grade stainless steel lining, thereby prolonging service life of a device, improving safety of a pressure vessel, reducing a heavy metal content of the product, and improving a product quality; and the deamination tower is used for removing the ammonia, thereby improving deamination efficiency and effect, and ensuring complete deamination. The present invention provides a new way for realizing efficient, environment-friendly, economical and safe continuous production of the glycine.

### BRIEF DESCRIPTION OF THE DRAWINGS

FIG. 1 is a process flowchart of a method in the example of the present invention.

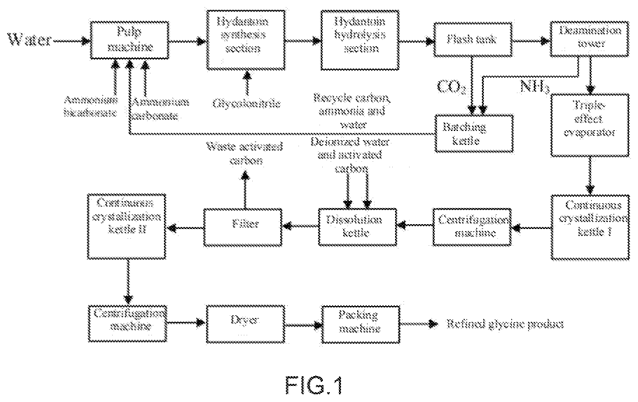

### DETAILED DESCRIPTION OF THE INVENTION

Disclosed is a kettle-type continuous production method for glycine. The method may be implemented by appropriately improving a process parameter by a person skilled in the art with reference to contents herein. It should be particularly pointed out that all similar substitutions and alterations are obvious to a person skilled in the art and are deemed to fall within the present invention. It is apparent that related personnel can make changes or proper alterations and combinations on the content herein without departing from the content, spirit and scope of the present invention to implement and apply a technique of the present invention.

In the present invention, scientific and technical terms used herein, unless otherwise indicated, generally have meanings commonly understood by a person skilled in the art. Part of the terms appearing in the present invention are explained below.

The term “glycolonitrile”, also known as glycolic nitrile, whose chemical formula and molecular weight are HOCH2CN and 57.05 respectively, is generally a colorless oily liquid, a hydrocyanic acid derivative, and the simplest cyanohydrin.

The term “glycine”, also known as an aminoacetic acid, whose chemical formula and molecular weight are C2H5NO2 and 75.07 respectively, is an amino acid having the simplest structure, and widely used in fields of pesticides, medicines, food, feed, daily chemicals, organic synthesis, etc.

The term “urea-grade stainless steel” is special austenitic stainless steel. Urea-grade stainless steel specially used for urea production mainly includes 316UG and 00Cr25Ni22Mo2N (2RE69). Urea is produced by synthesizing carbon dioxide and ammonia at a high pressure (140-250 atmospheric pressure) and a temperature of 180-210° C. An intermediate product such as ammonium carbamate has strong corrosion to the stainless steel, and general stainless steel such as 316L cannot meet corrosion resistance. Therefore, special steel developed is called the urea-grade stainless steel.

In order to enable a person skilled in the art to have a better understanding of a technical solution of the present invention, the present invention will be further described in detail below with reference to the specific examples.

### EXAMPLE 1

Raw materials were mixed in an amount-of-substance ratio of glycolonitrile to ammonium carbonate to ammonium bicarbonate to water being 1:1:2:20, and a slurry was input to a reactor with multiple serially connected kettles. The reactor included four serially connected reaction kettles with the same effective volume in total, where a first reaction kettle and a second reaction kettle formed a hydantoin synthesis section, and a third reaction kettle and a fourth reaction kettle formed a hydantoin hydrolysis section. Reaction temperatures, pressures and average retention times of material liquids of the four kettles were sequentially as follows: a temperature, a pressure and a retention time of the first reaction kettle were 80° C., 3 MPa and 1.5 h respectively, a temperature, a pressure and a retention time of the second reaction kettle were 100° C., 3 MPa and 1.5 h respectively, a temperature, a pressure and a retention time of the third reaction kettle were 130° C., 3 MPa and 1.5 h respectively, and a temperature, a pressure and a retention time of the fourth reaction kettle were 160° C., 3 MPa and 1.5 h respectively.

A reaction liquid output from the hydantoin hydrolysis section entered a flash tank to remove all carbon dioxide and parts of ammonia and water through two-stage flashing, then a flashed reaction liquid entered a deamination tower to remove all ammonia and part of water, removed carbon dioxide and ammonia were subjected to ratio adjustment by a batching kettle and then conveyed to a slurry machine for rebatching and recycling, then a reaction liquid entered a triple-effect evaporator for decompressing evaporation to remove a large amount of water to obtain a concentrated reaction liquid, and finally the concentrated reaction liquid was primarily crystallized, centrifugated, separated, heated, dissolved, decolored, filtered, secondarily crystallized, centrifugated and dried to obtain a refined glycine product.

The product was subjected to high performance liquid chromatography (HPLC) and inductively coupled plasma (ICP) for elemental analysis, and a result showed that the product had a content up to 99.5% without heavy metal and a yield of 99.1%. Energy consumption of an entire process was 920 kg standard coal/ton.

### EXAMPLE 2

A reactor with multiple serially connected kettles included four serially connected reaction kettles with the same effective volume in total, where a first reaction kettle and a second reaction kettle formed a hydantoin synthesis section, and a third reaction kettle and a fourth reaction kettle formed a hydantoin hydrolysis section.

Ammonium carbonate, ammonium bicarbonate and water were mixed in an amount-of-substance ratio of 2:3:30 to prepare a slurry, and the slurry was conveyed by a slurry pump to the hydantoin synthesis section of the reactor with multiple serially connected kettles, and subjected to a hydantoin synthesis reaction with glycolonitrile which was conveyed to the first reaction kettle and the second reaction kettle separately in a mass ratio of 3:1, where an amount-of-substance ratio of the glycolonitrile to the ammonium carbonate to the ammonium bicarbonate to the water was 1:2:3:30. Then a slurry entered the third reaction kettle and the fourth reaction kettle of the hydantoin hydrolysis section and was subjected to a hydantoin hydrolysis reaction. Reaction temperatures, pressures and average retention times of material liquids of the four kettles were sequentially as follows: a temperature, a pressure and a retention time of the first reaction kettle were 100° C., 7 MPa and 2.3 h respectively, a temperature, a pressure and a retention time of the second reaction kettle were 120° C., 7 MPa and 2.3 h respectively, a temperature, a pressure and a retention time of the third reaction kettle were 150° C., 7 MPa and 2.3 h respectively, and a temperature, a pressure and a retention time of the fourth reaction kettle were 180° C., 7 MPa and 2.3 h respectively. A reaction liquid output from the hydantoin hydrolysis section entered a flash tank to remove all carbon dioxide and parts of ammonia and water through two-stage flashing, then a flashed reaction liquid entered a deamination tower to remove all ammonia and part of water, removed carbon dioxide and ammonia were subjected to ratio adjustment by a batching kettle and then conveyed to a slurry machine for rebatching and recycling, then a reaction liquid entered a triple-effect evaporator for decompressing evaporation to remove a large amount of water to obtain a concentrated reaction liquid, and finally the concentrated reaction liquid was primarily crystallized, centrifugated, separated, heated, dissolved, decolored, filtered, secondarily crystallized, centrifugated and dried to obtain a refined glycine product.

The product was subjected to HPLC and ICP for elemental analysis, and a result showed that the product had a content up to 99.7% without heavy metal and a yield of 99.5%. Energy consumption of an entire process was 950 kg standard coal/ton.

### EXAMPLE 3

A reactor with multiple serially connected kettles included four serially connected reaction kettles with different effective volumes in total. The four reaction kettles were sequentially arranged in series from small effective volumes to large ones, where a first reaction kettle and a second reaction kettle formed a hydantoin synthesis section, and a third reaction kettle and a fourth reaction kettle formed a hydantoin hydrolysis section.

Ammonium carbonate, ammonium bicarbonate and water were mixed in an amount-of-substance ratio of 2:3:30 to prepare a slurry, and the slurry was conveyed by a slurry pump to the hydantoin synthesis section of the reactor with multiple serially connected kettles, and subjected to a hydantoin synthesis reaction with glycolonitrile which was conveyed to the first reaction kettle and the second reaction kettle separately in a mass ratio of 3:1, where an amount-of-substance ratio of the glycolonitrile to the ammonium carbonate to the ammonium bicarbonate to the water was 1:2:3:30. Then a slurry entered the third reaction kettle and the fourth reaction kettle of the hydantoin hydrolysis section and was subjected to a hydantoin hydrolysis reaction. Reaction temperatures, pressures and average retention times of material liquids of the four kettles were sequentially as follows: a temperature, a pressure and a retention time of the first reaction kettle were 100° C., 7 MPa and 0.8 h respectively, a temperature, a pressure and a retention time of the second reaction kettle were 120° C., 7 MPa and 1.3 h respectively, a temperature, a pressure and a retention time of the third reaction kettle were 150° C., 7 MPa and 2.0 h respectively, and a temperature, a pressure and a retention time of the fourth reaction kettle were 180° C., 7 MPa and 3.0 h respectively. A reaction liquid output from the hydantoin hydrolysis section entered a flash tank to remove all carbon dioxide and parts of ammonia and water through two-stage flashing, then a flashed reaction liquid entered a deamination tower to remove all ammonia and part of water, removed carbon dioxide and ammonia were subjected to ratio adjustment by a batching kettle and then conveyed to a slurry machine for rebatching and recycling, then a reaction liquid entered a triple-effect evaporator for decompressing evaporation to remove a large amount of water to obtain a concentrated reaction liquid, and finally the concentrated reaction liquid was primarily crystallized, centrifugated, separated, heated, dissolved, decolored, filtered, secondarily crystallized, centrifugated and dried to obtain a refined glycine product.

The product was subjected to HPLC and ICP for elemental analysis, and a result showed that the product had a content up to 99.8% without heavy metal and a yield of 99.7%. Energy consumption of an entire process was 940 kg standard coal/ton.

### EXAMPLE 4

A reactor with multiple serially connected kettles included four serially connected reaction kettles with different effective volumes in total. The four reaction kettles were sequentially arranged in series from small effective volumes to large ones, where a first reaction kettle and a second reaction kettle formed a hydantoin synthesis section, and a third reaction kettle and a fourth reaction kettle formed a hydantoin hydrolysis section.

Ammonium carbonate, ammonium bicarbonate and water were mixed in an amount-of-substance ratio of 1.5:2.5:25 to prepare a slurry, and the slurry was conveyed by a slurry pump to the hydantoin synthesis section of the reactor with multiple serially connected kettles, and subjected to a hydantoin synthesis reaction with glycolonitrile which was conveyed to the first reaction kettle and the second reaction kettle separately in a mass ratio of 5:1, where an amount-of-substance ratio of the glycolonitrile to the ammonium carbonate to the ammonium bicarbonate to the water was 1:1.5:2.5:25. Then a slurry entered the third reaction kettle and the fourth reaction kettle of the hydantoin hydrolysis section and was subjected to a hydantoin hydrolysis reaction. Reaction temperatures, pressures and average retention times of material liquids of the four kettles were sequentially as follows: a temperature, a pressure and a retention time of the first reaction kettle were 90° C., 5 MPa and 0.7 h respectively, a temperature, a pressure and a retention time of the second reaction kettle were 110° C., 6 MPa and 1.1 h respectively, a temperature, a pressure and a retention time of the third reaction kettle were 140° C., 4 MPa and 1.6 h respectively, and a temperature, a pressure and a retention time of the fourth reaction kettle were 170° C., 5 MPa and 2.8 h respectively. A reaction liquid output from the hydantoin hydrolysis section entered a flash tank to remove all carbon dioxide and parts of ammonia and water through two-stage flashing, then a flashed reaction liquid entered a deamination tower to remove all ammonia and part of water, removed carbon dioxide and ammonia were subjected to ratio adjustment by a batching kettle and then conveyed to a slurry machine for rebatching and recycling, then a reaction liquid entered a triple-effect evaporator for decompressing evaporation to remove a large amount of water to obtain a concentrated reaction liquid, and finally the concentrated reaction liquid was primarily crystallized, centrifugated, separated, heated, dissolved, decolored, filtered, secondarily crystallized, centrifugated and dried to obtain a refined glycine product.

The product was subjected to HPLC and ICP for elemental analysis, and a result showed that the product had a content up to 99.9% without heavy metal and a yield of 99.8%. Energy consumption of an entire process was 930 kg standard coal/ton.

### EXAMPLE 5

A reactor with multiple serially connected kettles included eight serially connected reaction kettles with different effective volumes in total. The eight reaction kettles were sequentially arranged in series from small effective volumes to large ones, where a first reaction kettle group (including a first reaction kettle and a second reaction kettle) and a second reaction kettle group (including a third reaction kettle and a fourth reaction kettle) formed a hydantoin synthesis section, and a third reaction kettle group (a fifth reaction kettle and a sixth reaction kettle) and a fourth reaction kettle group (including a seventh reaction kettle and an eighth reaction kettle) formed a hydantoin hydrolysis section.

Ammonium carbonate, ammonium bicarbonate and water were mixed in an amount-of-substance ratio of 2:3:20 to prepare a slurry, and the slurry was conveyed by a slurry pump to the hydantoin synthesis section of the reactor with multiple serially connected kettles, and subjected to a hydantoin synthesis reaction with glycolonitrile which was conveyed to the first reaction kettle group and the second reaction kettle group separately in a mass ratio of 5:1, where glycolonitrile was evenly assigned to four reaction kettles in the first reaction kettle group by mass, glycolonitrile was also evenly assigned to four reaction kettles in the second reaction kettle group by mass, and finally an amount-of-substance ratio of the glycolonitrile to the ammonium carbonate to the ammonium bicarbonate to the water was 1:2:3 20. Then a slurry entered the third reaction kettle group and the fourth reaction kettle group of the hydantoin hydrolysis section and was subjected to a hydantoin hydrolysis reaction. Reaction temperatures, pressures and average retention times of material liquids of the eight kettles were sequentially as follows: a temperature, a pressure and a retention time of the first reaction kettle were 90° C., 5 MPa and 0.7 h respectively, a temperature, a pressure and a retention time of the second reaction kettle were 90° C., 5 MPa and 0.6 h respectively, a temperature, a pressure and a retention time of the third reaction kettle were 110° C., 6 MPa and 1.1 h respectively, and a temperature, a pressure and a retention time of the fourth reaction kettle were 110° C., 6 MPa and 1.0 h respectively; and a temperature, a pressure and a retention time of the fifth reaction kettle were 140° C., 4 MPa and 1.6 h respectively, a temperature, a pressure and a retention time of the sixth reaction kettle were 140° C., 4 MPa and 1.5 h respectively, a temperature, a pressure and a retention time of the seventh reaction kettle were 170° C., 5 MPa and 2.7 h respectively, and a temperature, a pressure and a retention time of the eighth reaction kettle were 170° C., 5 MPa and 2.8 h respectively. A reaction liquid output from the hydantoin hydrolysis section entered a flash tank to remove all carbon dioxide and parts of ammonia and water through two-stage flashing, then a flashed reaction liquid entered a deamination tower to remove all ammonia and part of water, removed carbon dioxide and ammonia were subjected to ratio adjustment by a batching kettle and then conveyed to a slurry machine for rebatching and recycling, then a reaction liquid entered a triple-effect evaporator for decompressing evaporation to remove a large amount of water to obtain a concentrated reaction liquid, and finally the concentrated reaction liquid was primarily crystallized, centrifugated, separated, heated, dissolved, decolored, filtered, secondarily crystallized, centrifugated and dried to obtain a refined glycine product.

The product was subjected to HPLC and ICP for elemental analysis, and a result showed that the product had a content up to 99.9% without heavy metal and a yield of 99.9%. Energy consumption of an entire process was 930 kg standard coal/ton.

### Comparative Example 1

Glycolonitrile, ammonium bicarbonate and water were selected as raw materials and mixed in a molar ratio of 1:2:50, and a mixture was conveyed to a tubular packed reactor by a metering pump at a certain flow rate and subjected to a hydantoin synthesis reaction and a hydantoin hydrolysis reaction, where in the tubular packed reactor, a temperature, a pressure and a retention time of a first section were 90° C., 5 MPa and 15 min respectively, a temperature, a pressure and a retention time of a second section were 110° C., 5 MPa and 30 min respectively, a temperature, a pressure and a retention time of a third section were 130° C., 5 MPa and 30 min respectively, and a temperature, a pressure and a retention time of a fourth section were 160° C., 5 MPa and 1 h respectively. Then a material liquid entered a kettle type series reactor and was subjected to a hydantoin hydrolysis reaction continuously, where a temperature, a pressure and a retention time of a reaction kettle 1 were 160° C., 5 MPa and 2 h respectively, a temperature, a pressure and a retention time of a reaction kettle 2 were 170° C., 5 MPa and 1 h respectively, a temperature, a pressure and a retention time of a reaction kettle 3 were 190° C., 5 MPa and 0.5 h respectively, and a temperature, a pressure and a retention time of a reaction kettle 4 were 200° C., 5 MPa and 0.5 h respectively. Finally a material liquid was subjected to gas-liquid separation, decompressing distillation, crystallization refining, drying and dehydration to obtain refined glycine.

A product was subjected to HPLC and ICP for elemental analysis, and a result showed that the product had a content up to 99.6% without heavy metal and a yield of 99.3%. Energy consumption of an entire process was 1400 kg standard coal/ton.

The above descriptions are merely the preferred implementations of the present invention. It should be pointed out that those of ordinary skill in the art may also make several modifications and adaptations without departing from the principle of the present invention, and these modifications and adaptations shall fall within the scope of protection of the present invention.

What is claimed is:

1. A kettle-type continuous production method for glycine, the method comprising:
performing a hydantoin synthesis and hydrolysis reaction on glycolonitrile, ammonium carbonate, ammonium bicarbonate and water in a reactor with multiple serially connected kettles, and then purifying, concentrating, crystallizing, separating and drying the products to obtain refined glycine,
wherein the reactor with multiple serially connected kettles consists of a hydantoin synthesis section and a hydantoin hydrolysis section which are connected in sequence,
the hydantoin synthesis section comprising a first reaction kettle group of reaction kettles with a reaction temperature of 80-100° C. and a second reaction kettle group of reaction kettles with a reaction temperature of 100-120° C., the first reaction kettle group or the second reaction kettle group consisting of one or more reaction kettles connected in series,
the hydantoin hydrolysis section comprising a third reaction kettle group of reaction kettles with a reaction temperature of 130-150° C. and a fourth reaction kettle group of reaction kettles with a reaction temperature of 160-180° C., and the third reaction kettle group or the fourth reaction kettle group consisting of one or more reaction kettles connected in series.

2. The method according to claim 1, wherein the effective volumes of the reaction kettles of the reactor with multiple serially connected kettles are different, and the reaction kettles of the reactor with multiple serially connected kettles are sequentially connected from small effective volumes to the large ones.

3. The method according to claim 1 or 2, wherein the ammonium carbonate, the ammonium bicarbonate and the water are proportionally mixed into a slurry, and the slurry enters the reactor with multiple serially connected kettles, and is subjected to a synthesis reaction with glycolonitrile which is introduced to the hydantoin synthesis section.

4. The method according to claim 3, wherein a feeding mass distribution ratio of the glycolonitrile meets that a ratio of the total feeding amount of the first reaction kettle group to the total feeding amount of the second reaction kettle group is 3-5:1.

5. The method according to claim 3, wherein the ammonium carbonate, the ammonium bicarbonate and the water are preheated when proportionally mixed into the slurry.

6. The method according to claim 1 or 2, wherein an amount-of-substance ratio of the glycolonitrile to the ammonium carbonate to the ammonium bicarbonate to the water is 1:1-2:2-3:20-30.

7. The method according to claim 2, wherein a pressure and a retention time of the first reaction kettle group of reaction kettles are 3-7 MPa and 0.5-0.8 h respectively; a pressure and a retention time of the second reaction kettle group of reaction kettles are 3-7 MPa and 1.0-1.3 h respectively; a pressure and a retention time of the third reaction kettle group of reaction kettles are 3-7 MPa and 1.5-2.0 h respectively; and a pressure and a retention time of the fourth reaction kettle group of reaction kettles are 3-7 MPa and 2.5-3.0 h respectively.

8. The method according to claim 1 or 2, wherein the reaction kettle in the hydantoin hydrolysis section uses urea-grade stainless steel as a kettle body lining.

9. The method according to claim 1 or 2, wherein the purifying comprises removing, in a deamination tower, ammonia from a product of the hydantoin hydrolysis section.

10. The method according to claim 1, 2 or 4, wherein carbon dioxide and ammonia generated through the purifying, evaporation condensate generated through the concentrating and a crystallization mother liquid generated through the crystallizing are used as raw materials for recycling.
